(19) United States
(12) Patent Application Publication
(10) Pub. No.: US 2010/0137637 A1
(43) Pub. Date: Jun. 3, 2010

(54) CARBON-SUPPORTED GOLD CATALYST

(76) Inventors: Alireza Haji Begli, Ramsen (DE); Christine Kröner, Worms (DE); Nadine Decker, Erkner (DE); Ulf Prüsse, Braunschweig (DE); Klaus-Dieter Vorlop, Braunschweig (DE)

Correspondence Address: OSTROLENK FABER GERB & SOFFEN, 1180 AVENUE OF THE AMERICAS, NEW YORK, NY, 100368403, US

(21) Appl. No.: 12/525,170

(22) PCT Filed: Jan. 30, 2008
(86) PCT No.: PCT/EP2008/000694

§ 371 Date: Oct. 30, 2009

(30) Foreign Application Priority Data
Feb. 3, 2007 (DE) 10 2007 005 528.7

(51) Int. Cl. C07C 51/16; B01J 27/10; B01J 21/18; B01J 23/52

(52) U.S. Cl. 562/523; 502/181

(57) ABSTRACT

The invention relates to processes for preparing supported gold catalysts on carbon supports, wherein the support is contacted with aqueous solution or suspension of a chloroauric acid precursor. The invention also relates to a carbon-supported gold catalyst and to the use thereof for oxidation of alcohols, aldehydes, polyhydroxy compounds and carbohydrates.

### DESCRIPTION

The invention relates to processes for preparing supported gold catalysts with a porous carbon support and chloroauric acid precursor. The invention also relates to carbon-supported gold catalysts and to the use thereof for oxidizing especially alcohols, aldehydes, polyhydroxy compounds and carbohydrates.

### STATE OF THE ART

There is regularly a need for highly active and stable catalysts which can be used in particular for the oxidation of organic compounds such as alcohols, aldehydes, polyhydroxy compounds and mono-, oligo- and polysaccharides.

The use of supported palladium and platinum catalysts for oxidation of glucose is known. However, this is considerably restricted owing to the low selectivity and conversion rate. Reaction by-products can often be removed from the product mixture only with difficulty, if at all. The purity of a product is associated with the purifiability. Many reaction products are considered to be high-purity only because reaction by-products present cannot be removed. In some cases, the reaction by-products as such are not detectable or differentiable at all by common methods.

There is also frequently relatively rapid deactivation of the catalysts as a result of blocking of the catalyst surface owing to adsorption and/or owing to poisoning effects. This effect is known in particular for carbon as a support material; as is well known, activated carbon is used to purify product mixtures.

For industrial scale production of oxidation products from carbohydrates, fermentation processes are often still being used, which are associated with a high level of apparatus complexity and with wastewater pollution.

For this reason, novel catalyst types shall be developed, which enable effective catalytic oxidation, particularly of carbohydrates, for example for preparing aldonic acids using dioxygen as an oxidizing agent, and, as well as high activity and selectivity, also have a long lifetime.

Supported gold catalysts are known. They are used principally to oxidize CO or propene in the gas phase and for selective hydrogenations. Carbon-supported gold catalysts can also be used for selective oxidation of D-glucose to D-gluconic acid in the liquid phase. DE 103 199 17 A1 discloses supported gold catalysts with gold particles in nanodisperse distribution on carbon or metal oxide supports. These are used in particular for C1-selective oxidation of glucose and other carbohydrates. However, the activity of these catalysts is unsatisfactory.

Processes for preparing gold catalysts by impregnating the support by the “incipient wetness” method are known. However, the literature describes such impregnation methods as unsuitable for the synthesis of active and stable gold catalysts. This is in particular because purportedly only large gold particles (larger than 10 nm) are generally obtained in these processes.

### Objective

The technical problem underlying the present invention consists in providing improved supported gold catalysts and processes for preparation thereof, which have an improved activity and selectivity, in particular in the oxidation of organic compounds such as alcohols, aldehydes and polyhydroxy compounds. It is a further object of the invention to provide processes for selective and effective oxidation of carbohydrates, especially for preparing aldonic acids, which overcome the disadvantages of the prior art.

The underlying technical problem is solved by the provision of a process for preparing a supported gold catalyst according to claim 1, specifically from porous carbon support and chloroauric acid precursor, wherein the carbon support is provided in a step a), and the support is contacted with an aqueous solution or suspension of the chloroauric acid precursor in a step b). In step b), an impregnated catalyst precursor is obtained therefrom, and is dried in a subsequent step c). The process according to the invention is particularly characterized in that the support is provided in step a) in dry and preferably pulverized or granulated form or as a shaped body, and in that the volume of the aqueous solution or suspension of the chloroauric acid precursor is selected in step b) to be no greater than that which corresponds to the pore volume of the support. It can be selected to be smaller but not to be greater than the pore volume.

When the specific pore volume of the support is unknown, the volume of the aqueous precursor solution supplied to the dry support is preferably determined empirically by adding the precursor solution stepwise to the dry support until the support can no longer absorb any further volume of the precursor solution. This is recognizable in particular by the onset of a moist appearance of the support material. For any carbon support type, there is a specific absorbtivity [in ml/g_{catalyst support}] which depends in particular on the surface/volume ratio, on the pore size and on the degree of drying of the carbon support. “Dry” is understood to mean that the porous carbon support contains essentially no moisture in the pore volume, and so precursor solution can be absorbed into the pore volume.

In a particularly preferred variant, steps a) to c) are performed twice or more than twice in succession. In an alternative variant, steps b) and c) are performed simultaneously, i.e. parallel to one another in one reaction step.

In a preferred embodiment, the contacting of the carbon support with the chloroauric acid precursor in step b) takes place by dropwise addition of the precursor to the support with stirring. In a preferred variant, the precursor is sprayed onto the support, in the course of which the support is preferably stirred. Preference is given to drying during the stirring of the support with the applied precursor (step c)). In one variant, the contacting of the precursor with the support takes place in a coating tank or a pelletizing pan, in which case preference is given to dropwise addition or spray application and if appropriate simultaneous drying. In a further variant, the support is present in a fluidized bed and the precursor is introduced into the fluidized bed, preferably sprayed in; in this case, the support is preferably dried with the applied precursor (step c)).

The chloroauric acid precursor used is preferably an acidic solution or suspension of tetrachloroauric acid (HAuCl₄) in aqueous acid, especially hydrochloric acid, the concentration of the acid being preferably 0.1 mol/l to 12 mol/l, preferably 1 mol/l to 4 mol/l, more preferably 2 mol/l. In a particularly preferred embodiment, the pH of the finished precursor solution is always 6 or less, 5 or less, 4 or less, 3 or less, 2 or less and most preferably always 1 or less. Preferably or optionally—according to the application—the precursor solution used in accordance with the invention also comprises at least one further acid. Of course, it is possible to use further inorganic or organic acids as a further acid and instead of the hydrochloric acid.

Particular preference is given to preparing the aqueous precursor solution by directly weighing and dissolving the required amount of tetrachloroauric acid in the aqueous acid. Preference is given to dissolving the tetrachloroauric acid using aqueous hydrochloric acid preferably in a concentration of 0.1 mol/l to 12 mol/l, of 1 mol/l to 4 mol/l and more preferably of 2 mol/l.

TEM analyses have shown that the catalysts prepared in accordance with the invention surprisingly have very small and active particle sizes of less than 10 nm, especially of 1 nm to 10 nm, preferably of 1 nm to 9 nm, particularly of 1 nm to 5 nm or even of 1 nm to 2 nm. The inventors have succeeded for the first time with the process according to the invention in preparing catalytically active gold particles in sizes of significantly below 10 nm on a carbon support by the “incipient wetness” method. These results are surprising and are contrary to the descriptions or expectations of the relevant literature. The resulting gold catalysts exhibit an activity and selectivity which have not been achieved to date, for example in the conversion of glucose or lactose. Particularly through use of a strongly acidic precursor solution (for example 2 mol/l of HCl as the solvent for tetrachloroauric acid) and in the case of use of, it has been possible to prepare the most active carbon-supported gold catalyst to date. A catalyst prepared in accordance with the invention has, in glucose oxidation, an activity of about 2000 mmol gₘₑₜₐₗ⁻¹ min⁻¹.

HAuCl₄ is not stable in aqueous solution, but is hydrolyzed. A successive exchange of the chloride for water and hydroxyl groups takes place in a plurality of successive equilibria: [AuCl₄]⁻, [AuCl₃(OH)]⁻, [AuCl₂(OH)₂]⁻, [AuCl₂(OH)], [AuCl(OH)₂], [Au(OH)₃], [Au(OH)₄]⁻. These equilibria are time-and pH-dependent. A sufficiently low pH allows the hydrolysis to be prevented or influenced.

Without being bound to the theory, the tetrachloro complex [AuCl₄]⁻ dominates in strongly acidic aqueous solution (2 mol/l of HCl). The presence of this complex surprisingly leads to the effect that very small particles in particular are stabilized in the course of reduction of the catalyst precursor. In other, more weakly acidic solutions, there is probably a gradual successive exchange of the chloride ions for water and hydroxide ions.

In step c), preference is given to drying at temperatures of greater than or equal to room temperature, preferably of 60° C. to 200° C., more preferably of 60° C. to 100° C.

In a further step d), which is preferably performed after step c), the catalyst precursor is preferably reduced. This is preferably done in the hydrogen stream. The hydrogen stream preferably has a hydrogen content of 5% by volume to 15% by volume, preferably 10% by volume. According to the field of use, the hydrogen stream may optionally comprise at least one inert gas such as nitrogen or noble gas. More preferably, the hydrogen stream consists of hydrogen gas and at least one inert gas. Alternatively, the reduction can be effected as a liquid phase reduction in a manner known per se with suitable reducing agents such as sodium borohydride, formate salts, carbohydrates, formaldehyde or hydrazine.

When, in a preferred embodiment of the process according to the invention, steps a) to c), particularly steps b) and c), are performed repeatedly in succession, it is preferred that the catalyst precursor is reduced (step d)) intermediately, preferably after each passage through steps a) to c), particularly b) and c).

Preference is given to performing the reduction in step d) at temperatures of greater than or equal to 250° C. Preferably in accordance with the invention, the reduction is effected for 10 minutes to 300 minutes, preferably for 80 to 120 minutes.

The invention also envisages that at least one doping additive is added to the support and/or the aqueous solution or suspension of the chloroauric acid precursor. This doping additive is preferably selected from oxides of the alkali metals, of the alkaline earth metals and of the rare earth metals. Particular preference is given to doping with sodium, potassium, cesium, calcium, cerium and/or samarium.

Preference is given to adding the at least one doping additive in a proportion of 0.01% by weight to 1% by weight.

The present invention accordingly further also provides for the use of a chloroauric acid precursor which comprises a solution or suspension of tetrachloroauric acid (HAuCl₄) in a solvent, or consists thereof, the solvent being aqueous acid in a concentration of 0.1 mol/l to 12 mol/l, preferably of 1 mol/l to 4 mol/l, more preferably of 2 mol/l. The acid is preferably hydrochloric acid (HCl). The hydrochloric acid is preferably optionally present in conjunction with at least one further acid. According to the invention, this chloroauric acid precursor, for preparation of the carbon-supported gold catalyst, is preferably used according to one of the above-described processes.

The present invention further also provides a carbon-supported gold catalyst which is preparable or is prepared by the aforementioned process. The inventive catalyst is particularly characterized in that the mean size of the gold particles on the support is essentially less than 10 nm, preferably 5 nm or less, more preferably 1 nm to 2 nm. The inventive catalyst preferably has a gold content of 0.01% by weight to 10% by weight, preferably of 0.01% by weight to 2% by weight, more preferably of 0.3% by weight.

Finally, the present invention further provides for the use of the aforementioned inventive catalyst for oxidizing organic reactants, which are especially selected from alcohols, aldehydes and polyhydroxy compounds. Preferably in accordance with the invention, the catalyst is used in a heterogeneous catalysis. This means that the catalyst is present in solid form, while the reactants to be oxidized are present in fluid phase, for example as an aqueous solution. In that case, the dioxygen which is preferably used for the oxidation is sparged through the liquid phase as a gas and is distributed and dissolved in the liquid phase by intensive stirring. The catalyst is preferably used in the form of a powder or granule. In a further preferred variant, moldings are used, for example cylinders, hollow cylinders, spheres or extrudates.

In a preferred embodiment, an aqueous solution or suspension of the reactant or reactant mixture to be oxidized is prepared, which contains the reactant in a proportion of at least about 10 mmol/l, preferably at least about 100 mmol/l, 150 mmol/l, 200 mmol/l, 250 mmol/l, 1000 mmol/l or 1500 mmol/l. Subsequently, the preferably pulverulent inventive catalyst is added to the aqueous reactant solution in an amount of about 10 mg/l to 10 g/l, preference being given to using about 1 g of catalyst per liter. The ratio between the amount of the reactant(s) to be oxidized and the amount of the gold present on the carbon support is preferably at least about 300-400 000, more preferably at least 300, 500, 1000, 2000, 4000, 10 000, 20 000, 50 000, 100 000, 200 000 or 400 000.

Preference is given to performing the oxidation of the reactant or reactant mixture at a pH of 7 to 11, preferably of 8 to 10. Preference is given to using a temperature of 20° C. to 140° C., of 40° C. to 90° C. and more preferably of 40° C. to 80° C. The pressure is preferably about 1 bar to about 25 bar. Preference is given to sparging oxygen and/or air through the aqueous reactant solution of the reactant, the mixture or the composition at a sparging rate of 100 ml/(min×L_{reactor volume}) to 10 000 ml/(min×L_{reactor volume}), preferably of 500 ml/(min×L_{reactor volume}).

It is found that a 100% selectivity for the aldehyde position occurs with the inventive gold catalysts in the oxidation of aldoses. The inventive gold catalysts are therefore also suitable for the selective oxidation of carbohydrates. This is understood to mean especially the oxidation of an oxidizable aldehyde group on the Cl carbon of a carbohydrate to a carboxyl group, whereas alcohol groups on other carbon atoms of the carbohydrate are not oxidized. As a result, therefore, aldonic acid is preferably obtained. The carbohydrates used with preference in accordance with the invention are preferably aldoses which have an oxidizable aldehyde group on the Cl carbon, or 2-ketoses in which an oxidizable aldehyde group can be introduced at the Cl carbon atom. The selective oxidation of the aldehyde group of an aldose affords an aldonic acid. The selective oxidation of a mixture of aldoses therefore affords a mixture of different aldonic acids.

The present invention therefore also relates to the use of the inventive catalysts for preparing an aldonic acid or a mixture of different aldonic acids by selective oxidation of one or more aldoses with an oxidizable aldehyde group.

The present invention therefore also relates to the use for preparing an aldonic acid or a mixture of different aldonic acids using one or more 2-ketoses, said 2-ketose(s) first being converted to the tautomeric aldose form(s) with an oxidizable aldehyde group and then being oxidized selectively using the catalyst.

According to the invention, the carbohydrates to be oxidized include monomeric polyhydroxyaldehydes or polyhydroxyketones, i.e. monosaccharides, the dimers to decamers thereof, i.e. oligosaccharides such as disaccharides, trisaccharides, etc., and the macromolecular polysaccharides. In the context of the present invention, “monosaccharides” are understood to mean compounds of the general chemical formula CₙH₂ₙOₙ with 3 to 7 oxygen functions, natural monosaccharides being essentially hexoses and pentoses. The carbon chain of a monosaccharide may be unbranched or branched. “Oligosaccharides” are understood to mean compounds which are obtained by combining 2 to 10 mono-saccharide molecules with loss of water.

The catalyst is more preferably used for selective oxidation of carbohydrates selected from monosaccharides such as glucose, galactose, mannose, xylose and ribose, and disaccharide aldoses such as maltose, lactose, cellobiose and isomaltose, and also disaccharide 2-ketoses such as palatinose, and also starch syrups and maltodextrins, and mixtures of these carbohydrates. Owing to the high selectivity, it is possible to directly oxidize typical starch syrups, known as industrial syrups.

The oxidation of glucose using the process in accordance with the invention affords gluconic acid as the oxidation product. The oxidation of galactose using the process according to the invention affords galactonic acid as the oxidation product.

In a further preferred embodiment, the carbohydrate to be oxidized is an oligosaccharide, especially a disaccharide. The disaccharide to be oxidized is preferably a disaccharide aldose such as maltose, lactose, cellobiose or isomaltose. According to the invention, the selective oxidation of maltose using the process according to the invention affords maltobionic acid as the oxidation product. Using the process according to the invention, the lactose oxidation affords by-product-free lactobionic acid as the oxidation product.

In a further preferred embodiment of the invention, the oligosaccharide to be oxidized is a disaccharide ketose. The disaccharide ketose to be oxidized is preferably palatinose (isomaltulose). Before the oxidation, palatinose is converted in accordance with the invention to the tautomeric aldose form, which is then oxidized.

In a further preferred embodiment of the invention, the carbohydrate to be oxidized is a maltodextrin. Maltodextrins are water-soluble carbohydrates obtained by enzymatic starch degradation, especially dextrose equivalents, with a chain length of 2 to 30 and preferably 5 to 20 anhydroglucose units, and a proportion of maltose. The selective oxidation of maltodextrin using the process according to the invention affords an oxidation product which, in accordance with the invention, according to the composition, has a proportion of maltobionic acid and gluconic acid in addition to the oligosaccharide aldonic acids.

In a further preferred embodiment, the carbohydrate to be oxidized is a starch syrup. A starch syrup is understood to mean a glucose syrup which is obtained from starch and in particular is present as a purified aqueous solution, the dry mass generally being at least 70%.

In a further preferred embodiment, the carbohydrate to be oxidized is a furfural. The furfural to be oxidized is preferably hydroxymethylfurfural (HMF) or glycosyloxymethyl-furfural (GMF).

### WORKING EXAMPLES

The invention is illustrated in detail by the examples which follow, though the examples should not be interpreted in a restrictive sense.

### Example 1

### Catalyst Preparation

### Preparation of the Chloroauric Acid Precursor

The required amount of tetrachloroauric acid in crystalline form (from Chempur (50% Au)) is dissolved in that volume of a solvent which corresponds to no more than the pore volume of the amount of support used.

Various catalysts were prepared, in which the HAuCl₄ precursor was dissolved in hydrochloric acid, water and potassium hydroxide solution. In addition, an aqueous solution of the precursor (25 g/l of Au) which had been stored for a prolonged period was appropriately diluted with water and hydrochloric acid. The following batches of chloroauric acid precursor were prepared:

1. Precursor weighed in and dissolved in 2 mol/l of HCl
2. Precursor solution diluted from aqueous precursor stock solution at 0.2 mol/l of HCl
3. Precursor weighed in and dissolved in water
4. Precursor solution diluted from aqueous precursor stock solution with water
5. Precursor weighed in and dissolved in aqueous KOH

In order to obtain catalysts with a different gold content, each batch was made up or diluted repeatedly in different concentrations in each case. The intention was to prepare gold catalysts with metal contents between 0.1 and 5%. 2 g of gold catalyst were prepared in each case per batch.

### Impregnation of the Carbon Supports, Incipient Wetness Method

The precursor solutions were gradually added dropwise to the support material with simultaneous intensive mixing in separate batches in each case. The end of the addition is recognizable by the onset of moisture on the support material, which indicates the saturation of the pore volume and hence the limit of the absorption capacity of the support.

### Drying, Reduction

The impregnated catalyst precursors were dried overnight in a drying cabinet (approx. 80° C.) and then reduced at 250° C. in a nitrogen/hydrogen stream (approx. 10% H₂) for 3 h. This is followed by cooling in a nitrogen stream.

### Results

### a) Gold Content

For all gold catalysts prepared, the gold content was first determined by means of ICP-AES. Gold catalysts with metal contents between 0.1 and 5% were prepared. The experimentally determined gold contents are compared with those calculated theoretically. The theoretical gold contents and actual gold contents have excellent correlation in all batches. It is possible to apply the gold to the support without loss.

### b) Particle Size

The TEM analysis of the gold catalysts shows particle sizes of 1 to a maximum of approximately 10 nm.

### c) Reduction Temperature

Profiles of the temperature-programmed reduction (TPR profiles) of all catalysts were recorded in each case. The highest reduction temperature is exhibited by the catalyst for which the precursor was weighed in in strongly acidic solution: 2 mol/l of HCl; the lowest is exhibited by the catalyst for which the precursor solution was diluted with water.

Strong adsorption of the gold precursor onto the support can be concluded from a high reduction temperature.

### Example 2

### Catalytic Oxidation of Glucose

The catalytic performance of the catalysts prepared according to example 1 was tested in the liquid phase oxidation of glucose to gluconic acid. The reaction was performed in a temperature-controlled glass reactor (volume 500 ml) at 40° C. The sparging was effected through a glass frit with an oxygen flow rate of 500 ml/min. The starting glucose concentration was 100 mmol/l. The pH was kept constant at pH 9 with the aid of a titrator (Titroline alpha, from Schott) and 2 mol/l of potassium hydroxide solution. Since gluconic acid is a monocarboxylic acid, the amount of acid formed can be concluded directly from the volume of alkali consumed with 100% selectivity. There was additionally a check by means of HPLC.

### Results

### a) Selectivity

In this reaction, the gold catalysts prepared exhibit 100% selectivity for the aldehyde position (C1) of glucose.

### b) Catalytic Activity

The conversion was complete (100%) for all reactions. For comparison of the catalysts, the maximum specific activity was employed.

### c) Long-Term Stability

The study of the long-term stability showed that the catalysts possess excellent long-term stability. No gold leaching was observed. The activity increase with increasing number of tests is case attributable to a reduced oxygen limitation as a result of catalyst loss.

1. A process for preparing a supported gold catalyst composed of porous carbon support and chloroauric acid precursor, comprising the steps of:
a) providing the dry support,
b) contacting the support with a solution or suspension of the precursor HAuCl₄ in the form of the tetrachloro complex, the volume of the precursor solution being less than or equal to the pore volume of the support, to obtain an impregnated catalyst precursor, and
c) drying the impregnated catalyst precursor,
said precursor solution being a solution or suspension of HAuCl₄ in aqueous acid and the pH of the finished precursor solution always being less than 1.

2. The process according to claim 1, wherein the precursor solution is added to the dry support in step b) stepwise and overall only in the volume for which the support cannot absorb any further volume of the solution.

3. The process according to claim 1, wherein the precursor solution is a solution or suspension of HAuCl₄ in 0.1 mol/1 to 12 mol/l aqueous hydrochloric acid, optionally in conjunction with at least one further acid.

4. The process according to claim 1, wherein the catalyst precursor is reduced in a further step d) in a hydrogen stream, at temperatures of greater than or equal to 250° C. or by means of liquid phase reduction.

5. The process according to claim 4, wherein the reduction in step d) is effected for a period of 10 min to 300 min.

6. The process according to claim 4, wherein the hydrogen stream in step d) comprises a hydrogen content of 5% by volume to 15% by volume and optionally inert gas.

7. The process according to claim 4, wherein the drying in step c) is effected at 60° C. to 200° C.

8. The process according to claim 1, wherein at least one doping additive selected from oxides of the alkali metals, alkaline earth metals and rare earth metals is present in a proportion of 0.01% by weight to 1% by weight in at least one of the carbon support and in the precursor solution.

9. (canceled)

10. A carbon-supported gold catalyst prepared by the process according to claim 1.

11. The catalyst according to claim 10, wherein the mean size of the gold particles on the support is less than 10 nm.

12. The catalyst according to claim 10, wherein the gold content is from 0.01% by weight to 10% by weight.

13. A method for oxidizing organic compounds selected from alcohols, aldehydes, carbohydrates and polyhydroxy compounds, said method comprising oxidizing said organic compounds with a carbon-supported gold catalyst according to claim 10.

14. The method according to claim 13 wherein the method comprises a selective oxidation of carbohydrates selected from monosaccharides, disaccharide aldoses, disaccharide 2-ketoses starch syrups, and maltodextrins, and mixtures thereof.

15. The method according to claim 14, wherein the monosaccharides are selected from glucose, galactose, mannose, xylose, ribose and mixtures thereof.

16. The method according to claim 14, wherein the disaccharide aldoses are selected from maltose, lactose, cellobiose, isomaltose and mixtures thereof.

17. The method according to claim 14, wherein the disaccharide 2-ketose is palatinose.
